(19) United States
(12) Patent Application Publication
(10) Pub. No.: US 2026/0272572 A1
(43) Pub. Date: Sep. 17, 2026

(54) NAVIGATION SYSTEM AND NAVIGATION METHOD HAVING AN ANNOTATION FUNCTION

(71) Applicant: B. Braun New Ventures GmbH, Freiburg im Breisgau (DE)

(72) Inventor: Peter Heinze, Karlsruhe (DE)

(21) Appl. No.: 18/997,829

(22) PCT Filed: Jul. 25, 2023
(86) PCT No.: PCT/EP2023/070610

§ 371 Date: Jan. 23, 2025

(30) Foreign Application Priority Data
Jul. 28, 2022 (DE) 10 2022 118 990.2

(51) Int. Cl. A61B 34/30; A61B 34/20; A61B 90/00; G16H 30/40; H04N 13/239; H04N 13/246; H04N 13/361

(52) U.S. Cl. CPC A61B 34/30; G16H 30/40; H04N 13/239; H04N 13/246; H04N 13/361; A61B 2034/2055; A61B 2034/2065; A61B 2090/364

(57) ABSTRACT

A surgical navigation system has an annotation function for inputting medical annotations and outputting medical annotations superimposed on visual images during a surgical procedure. The system includes a display for displaying medical annotations and a robot with a stereoscopic recording system for 3D recording of a structure or surface. A tracking system detects and tracks the position of the recording system and provides the position in computer-readable form. An annotation system has a storage unit that stores an annotation database with a global coordinate system for annotation objects. A control unit processes the 3D recording, determines a position of the 3D recording in the global coordinate system, and outputs an annotation object of the annotation database in the 3D recording via the display device in a superimposed display. The system can be used in a navigation method that is executable with instructions stored in a computer-readable storage medium.

### CROSS-REFERENCE TO RELATED APPLICATIONS

This application is the United States national phase entry of International Application No. PCT/EP2023/070610, filed on Jul. 25, 2023, and claims priority to German Application No. 10 2022 118 990.2, filed on Jul. 28, 2022. The contents of International Application No. PCT/EP2023/070610 and German Application No. 10 2022 118 990.2 are incorporated by reference herein in their entireties.

### FIELD

The present invention relates to a surgical navigation system having an annotation function/a marker function for inputting position-related, in particular visual, medical annotations/markers or recordings and/or for outputting medical annotations/markers during a surgical procedure on a patient, comprising: a (visual) display device, in particular a surgical monitor or a display or AR glasses or VR glasses, for the visual output of the medical annotations; and at least one robot, preferably plural robots actuatable independently of each other, as a positioning system (each) having a, in particular multi-link, robot arm movably connected to a robot base, and a robot head arranged at the end of the robot arm. A module to be positioned, in particular an end effector such as an instrument, can be connected to the robot head. In addition, the present disclosure relates to a navigation method as well as to a computer-readable storage medium and to a computer program.

### BACKGROUND

In the field of surgical navigation, currently there exist in general substantially two different groups/kinds/types/classes of surgical navigation systems.

The first group of the surgical navigation systems makes use of recordings/images such as computerized tomography recordings (CT recordings), magnetic resonance imaging recordings (MRI recordings), X-rays, ultrasonic scans which are generated preoperatively, viz. prior to the actual surgical procedure on the patient, a so-called image-based navigation. The preoperative recordings have to be correlated with/compared to the position or pose of the patient during the surgical procedure to enable appropriate navigation of instruments or implants. In so doing, the patient is registered relative to the preoperative recordings to correlate the virtual anatomy of the patient of the preoperative (3D) recording data with the intraoperative (current) real anatomy and to display instruments relative to the preoperative recording data.

The second group of the surgical navigation systems works without any recordings or images (imageless navigation). These navigation systems produce a model of the patient during the surgical procedure, i.e., intraoperatively, usually by scanning anatomic landmarks/reference points by a navigation probe/a pointer/a navigation pointer or by a kinematic analysis with corresponding simplified axis models.

The first group of the navigation systems, that is, the image-based navigation systems, have the drawback of a complex and time-consuming registration which frequently does not provide the required precision that is required for the surgical procedure, however.

In contrast to that, imageless navigation systems require no preoperative imaging/recordings and no intraoperative registration, but have the drawback of using only a very rudimentary model of the anatomy of the patient as a reference, such as an axis model of a leg in the case of knee surgery.

For an orthopedic navigation, there are current efforts to combine the advantages of both technologies, i.e., to do without preoperative imaging and intraoperative registration, but to have an as precise recording of the patient and/or the patient's surgery area as possible as a reference to appropriately align instruments or implants and to finally achieve the best result for the patient.

The image-based navigation methods currently are the standard in modern computer-based surgery. One central part of the image-guided/image-based navigation is a computerized tomography visualization/CT visualization of target positions, target trajectories and the display of CT risk objects/structures with respect to the preoperatively detected medical recording data. This (guide) information is currently derived solely from the preoperatively detected medical image data, that is, in terms of time before the procedure, and are used as a primary guiding modality during specific steps of the procedure.

Usually, for the image-based navigation an optical tracking system (as measurement system), in particular an active or passive optical infrared system, or an electromagnetic tracking system is used to align the preoperative recordings and/or the information thereof about arranged markers, in particular reflecting reference points, on all necessary objects with the surgery area/surgical site. Consequently, the visualization of said preoperative information with respect to the current intraoperative microscopic or endoscopic (current) live view of the patient's surgery area/surgical field unfortunately is very unprecise. In particular, such imprecision can be based on an unprecise registration. A particularly high imprecision may be due to the fact that an anatomy of the patient is shifted between a preoperative (image) recording and an intraoperative situation, as this is the case, for example, in a so-called brain shift when a patient's skull is opened.

In particular, the following aspects affect the imprecision or precision of the navigation: a tracking error for each individual tracked rigid body/fiducial set); an error of the patient's registration; an error in reconstructing the recording; a change of the anatomy with respect to the preoperative recording data such as by a shift of tissue just as a so-called brain shift; an error in a hand-eye camera calibration; and/or an error in an optical (camera) calibration.

In addition, the optical tracking technologies also require a (continuous) line of view between the tracking camera, on the one hand, and an instrument to be tracked, on the other hand, which in turn strongly limits the actually possible working area of the surgeon. For example, an unfavorable position of the surgeon or of other instruments or a robot arm may conceal the view and/or interrupt the line of view, resulting in an undesired interruption of the tracking during the surgical procedure and endangering the patient's safety. Also, the duration of the surgical procedure will be increased.

### SUMMARY

Therefore, the targets and the object of the present disclosure are to eliminate or at least to reduce the drawbacks of prior art and, in particular, to provide a surgical navigation system, a navigation method, a computer-readable storage medium and a computer program having an annotation function which offers improved navigation, particularly higher precision of navigation with improved flexible annotation. Another partial object can be perceived in collecting and centrally depositing information from different sub-systems and in appropriately centrally integrating said information for a navigation in order to be able to make organized use of all data. In particular, it is a partial object to provide a navigation with high precision intraoperatively during the surgical procedure even without any preoperative information (recordings).

Hence, a core idea of the present disclosure is the current intraoperative detection (viz. during the surgical procedure on the patient) of characteristic points or objects in a recording, in particular a 3D recording, and a corresponding registration (or position recognition, in particular pose recognition of the points, objects or areas of tissue structures) and storage with at least the corresponding coordinates (X, Y and Z coordinates) in one (single) central global coordinate system. The global coordinate system can be aligned relative to the patient, e.g., as a coordinate system of the patient, or alternatively can be aligned with the navigation system, in particular with a zero point of the coordinate system (COS) of the tracking system, preferably a zero point in a tracking camera. It is only crucial that all the information is transferred into one single central coordinate system so that a later conversion as well as sources of error are eliminated.

The determination of the (position) coordinates of the annotation object incorporates the different position data which are included by a combination of navigation (global position detection), a visualization by the recording system (local position detection) and of the robot or the robotics (sensory position detection via the robot arm). In other words, according to the present disclosure, an improvement of the navigation can be achieved by a combination of visualization, robotics and navigation.

In yet other words, there is provided a surgical navigation system having an annotation function/marking function for inputting position-related, in particular visual, or else nerve signal or histology information at a particular point of the anatomy, medical annotations or recordings and/or for outputting medical annotations during a surgical procedure on a patient, comprising: a display device, in particular a surgical monitor, for the visual output of the medical annotations; a robot as a positioning system comprising a, in particular multi-link, robot arm movably connected to a robot base, and a robot head arranged at the end of the robot arm, the navigation system further comprising: a stereoscopic recording system arranged on or in the robot head, in particular a stereo camera, particularly preferred a stereo microscope, comprising at least two recording units spaced apart from each other in order to perform a (current) 3D recording of a structure or surface (of a target area) relative to the recording system and to provide the latter together with recording parameters (of the recording system) in a computer-readable form; a tracking system adapted to detect and track, in particular indirectly via the pose (viz. the position and the orientation) of the robot head, the pose of the recording system and to provide the pose of the recording system in a computer-readable form; an annotation system/marking system having a storage unit in which a digital annotation database/marking database with a global coordinate system is stored/deposited; and a control unit adapted to process the provided 3D recording together with the recording parameters and the provided pose and, based on the provided recording parameters and the pose of the recording system, to determine a pose of the 3D recording in the global coordinate system and to output at least one annotation object of the annotation database (which is also stored in the global coordinate system and thus is correlated with the pose of the 3D recording) in the 3D recording at least in the correct position, in particular in the correct pose, in a superimposed display via the display device and to improve navigation.

Consequently, in particular relative to the (current intraoperative) 3D recording at any time and at any position of the recording system, the stored annotation object and/or the annotation(s) can be displayed superimposed on the 3D recording in the correct position, in particular in the correct pose, (if the position, in particular the pose, of the annotation is in the field of view of the recording system, viz. in the 3D recording).

In particular, the control unit can be adapted to determine a pose of the 3D recording for an annotation instruction/marking instruction (input by a user) based on the provided recording parameters and the pose of the recording system, and to store, as an annotation object/marking object, at least a section or a target area of the 3D recording together with the recording parameters in the annotation database of the annotation system in the global coordinate system in the determined position, in particular pose, in order to output, at a later point in time, the annotation object via the display device in a superimposed display in the correct position, in particular in the correct pose, and to improve navigation. By said navigation system, annotations can be centrally deposited with particularly high precision as regards the position data, in particular the pose data.

In yet completely other words, a novel surgical navigation system is provided which comprises one or more stereoscopic recording systems/optical camera systems which are attached to or arranged on a positioning system (in particular a robot arm, an optical tracking system, an electromagnetic system). The (primary) recording system is used intraoperatively (i.e., during the surgical procedure) to display and/or visually output a (current) (3D) recording such as a live 3D video view of the surgical site on the display device. Image-internal or spatial annotations/notes can be set manually or automatically by additional tracking systems (measuring systems or tracking systems) on the live video image of the surgery field/surgical area. Said image-internal or spatial annotations are detected and stored in the same (single) global coordinate system and, therefore, can be visualized and reconstructed by the user if needed for surgical guide. This is possible with high reproducibility and higher overall precision, even if the positioning system (robot arm, optical tracking system, electromagnetic system) was moved in the meantime and the camera parameters, such as a focal point or a focal plane, were changed. The term “focal point” defines a point in space which is located in a focal plane of the optical system of the recording system and/or the recording unit and, in particular on the optical axis.

In particular, a robot kinematics-based pose determination of the stereoscopic recording system in the coordinate system of the robot is performed, e.g. via the robot, and a pose determination of the 3D recording in the coordinate system of the recording system is performed via the recording system. Said two pose determinations are connected to each other by transforming, on the basis of the determined pose of the recording system in the robot coordinate system, for example, the determined pose of the 3D recording into the robot coordinate system in order to finally determine, similarly to a serial transformation, the pose of the 3D recording relative to the robot (and, resp., the robot coordinate system thereof). After that, said pose of the 3D recording is determined, for example by a defined or determinable transformation from the robot coordinate system to the global coordinate system (in one embodiment this can be the (virtual) coordinate system of the patient/patient coordinate system), in the global coordinate system. In other words, from different modalities data for an annotation can be obtained which are transferred or transformed into a single global coordinate system and can be stored there with the corresponding position (with X-Y-Z coordinates in space), in particular the corresponding position and orientation (viz. pose), in the global coordinate system. In this way, there are no longer provided different annotations in different databases with different (local) coordinate systems, but the annotations are integrated into a joint database with a joint global coordinate system. Thus, virtually a standardization is carried out and a uniform system is created by the centrally deposited annotations (for example, a database having standardized annotations in the form of a data array having: annotation ID, annotation position, annotation orientation, annotation data (such as an image, vector graphics, a text or the like) and optionally an annotation transparency (0% to 100%, at 100% annotation is switched off/not visible)). Said annotations then can be selected and rendered, in particular computer-based, so that they are shown to be superimposed in a superimposed display with a 3D recording of the recording system in the correct position (i.e., at the correct position relative to the 3D recording and thus to the patient), in particular in the correct pose (i.e., at the correct position in the correct orientation). If, in particular, the patient is tracked and registered, it is advantageous to utilize a patient coordinate system which is appropriately correlated as a global coordinate system. In this way, different annotations of the central database with a uniform global coordinate system can be displayed in a 3D recording, for example as a non-transparent or semi-transparent superposition.

In the present case, in particular a surgical navigation system is described which has at least one stereoscopic optical recording system attached to or arranged on a positioning system (in particular a robot arm) which produces an intraoperative 3D recording in order to show a live video view (live 3D recording) of the surgical site. Further cameras can be additionally used to detect further spatial and visual information from the surgical field. The recording system attached to the positioning system (in particular robot arm, optical tracking system, electromagnetic tracking) can be moved by the user, such as a surgeon, to specific spatial positions and can be aligned, in particular regarding the orientation, the recording system providing the user with a live view onto the surgical field.

The surgeon then can either make use of a manual method to identify and to label different anatomic or physiological regions, for example in the output 3D recording can set a point as annotation by a mouse pointer, or additional measuring systems and methods can be applied to automatically annotate, in the present case to label particularly appropriate regions. Accordingly, at least one 3D recording, in particular a live video image (annotation reference recording), as well as the spatial position thereof (coordinates) via the positioning system (in particular via the current robot arm configuration) and current camera settings are transferred together as annotation object into in the global coordinate system and are stored.

In contrast to existing waypoint functionalities in robot-based microscopes or annotations in microscope systems, the present navigation system stores both more and more precise data and features for an accurate and convenient visualization of the annotation objects with respect to the patient's anatomy. Further, the navigation system offers the possibility to move the whole navigation system or to modify the camera settings during the surgical procedure and nevertheless to reconstruct and to visualize the annotation objects. Due to the disclosed navigation system changes of the anatomic structures can be safely recognized, as the data are detected intraoperatively and not preoperatively.

The term “tracking system” describes a technical system that allows for a spatial localization and can detect a position and/or orientation.

The term “position” means a geometrical position in the three-dimensional space, which is indicated in particular using coordinates of a cartesian coordinate system. In particular, the position can be indicated by the three coordinates X, Y and Z.

The term “orientation”, in turn, indicates an alignment (e.g. with the position) in space. It can also be said that an alignment with a direction and/or rotation in the three-dimensional space is indicated by the orientation. In particular, the orientation can be indicated by means of three angles.

The term “pose” includes both a position and an orientation. In particular, the pose can be indicated by means of six coordinates, three position coordinates X, Y and Z and three angular coordinates for the orientation.

The term “in the correct position” defines that the annotation and/or the 3D recording is reproduced at the actual correct position in the superimposed display, similarly to a CAD system.

The term “in the correct pose” defines the combination of in the correct position and in the correct orientation in which the recording is shown both at the correct position and in the correct orientation in the superimposed display. In the case of a parallel coherent alignment of the recording directions, the superimposed recording is displayed with constant side ratios just as in the actual recording. If the recording direction is angled, for example, an extension or a dimension of the recording of the related direction is compressed corresponding to the angle, until with an angle of 90° between the two recording directions only a one-dimensional line is produced in the superimposed display.

The term “target area” describes a part of a surgical area which is of interest and is to be envisaged by the recording system. In particular, the target area can be one single target point in space (with X, Y, Z coordinate) and can also be indicated as such coordinate. However, the target area can also be a section from a two-dimensional plane, such as a small section of a tissue surface. Also, a target area can be indicated as a small cuboidal or spherical volume.

In contrast to an image-based navigation, the suggested navigation system and navigation method only have the following negative influencing factors on the precision: hand-eye calibration between the camera and the robot base coordinate system; and a calibrated camera system, which have to be taken into account and can be kept appropriately precise.

Advantageous embodiments will be explained in particular as follows.

According to one embodiment, a robot arm configuration of the robot and a camera setting of the recording system can be stored as recording parameters in the annotation database in order to move/displace the robot at a later point in time of the surgical procedure into exactly said position and orientation with a corresponding camera setting and in order to output, in the superimposed display, the current 3D recording and the previously stored 3D recording as a coherent reference, for example as a juxtaposed display or as a transparent superimposed display to visualize a change over time. In particular, for a so-called intra-image annotation, viz. an annotation inside the image, the camera of the recording unit has to calibrated, and for the repeated visualization this technique requires the recording system to be set to the same zoom and the same focus as during the annotation process (coherent view).

In particular, as recording parameters a zoom (factor) and a focus can be stored detected as camera settings of the stereo camera so that at least the position of a segment of the 3D recording is precisely determinable and/or is also retrievable for a visualization via the pose of the recording system tracked by the tracking system and via the zoom as well as the focus.

Further, the control unit can preferably be adapted, in the case of an output instruction concerning an annotation object stored in the annotation database, to move/displace the recording system into the pose stored relative to the annotation object and to output, in the superimposed display, the current 3D recording and the stored annotation object, in particular the section of the stored 3D recording, via the display device.

In particular, the annotation, in particular the intra-image annotation, can also be supported by computer vision algorithms by tracking corresponding regions while the zoom step and the focus/focal length are varied.

According to another preferred embodiment, in the storage unit further preoperative 3D recording data, in particular CT recording data and/or MRI recording data and/or segmented X-rays, can be stored, the surgical navigation system can be adapted to register the patient relative to the preoperative 3D recording data, and the control unit can be adapted to display also the preoperative 3D recording data in addition to the 3D recording in the superimposed display for further support. In other words, medical preoperative recording data/imaging data (3D CT data/3D MRI data) can thus be additionally used as supportive information which are not displayed as primary guiding information, however, but as secondary guiding information only.

The primary guiding information is constituted by the intraoperative recordings and the associated annotations, respectively.

According to another embodiment, the control unit can be adapted, when the section of the 3D recording stored in the annotation database relative to the current 3D recording, in particular by means of an image analysis method, deviates beyond a preset tolerance, to display a warning message in the superimposed display.

In particular, the control unit can be adapted to determine, if the 3D recording stored in the annotation database deviates from the current 3D recording, in particular by means of an image analysis method, beyond a preset tolerance, that there is a shift of the entire patient, if the stored 3D recording or at least the section of the 3D recording, is shifted as a whole relative to the current 3D recording, and preferably the control unit can additionally be adapted to newly register the navigation system relative to the patient, in particular to correct the registration by exactly the established shift.

Furthermore, in particular the control unit can be adapted to determine, in the case of deviation of the 3D recording stored in the annotation database relative to the current 3D recording, in particular by means of an image analysis method, beyond a preset tolerance, that there is a shift of only a tissue of the patient, if the stored 3D recording or at least the section of the 3D recording is distorted, such as compressed, relative to the current 3D recording, and in addition to preferably perform a registration relative to the relevant tissue.

In particular, the control unit can be adapted to detect an instrument in the current 3D recording and to put/display the same in the superimposed display into a front plane, while the at least one annotation object is displayed in an intermediate plane between the live recording and the instrument, i.e., is shown in a rear plane viewed relative to the instrument, so that the annotation objects do not interfere with the instrument and/or the instrument is not covered by the annotation objects in the superimposed display. Moreover, in particular the control unit can be adapted to detect an instrument in the current 3D recording and to put/display the same into a rear plane in the superimposed display, while the at least one annotation object is shown in a front plane, i.e., in a front plane viewed relative to the instrument. Thus, annotations may be located in front of or behind the instrument. When they are located in front of the instrument, they cover the instrument in a non-transparent or semi-transparent manner (e.g. 70% coverage), and when the annotation objects are located behind the instrument, the instrument covers the annotation in a non-transparent or semi-transparent manner (e.g. 70% coverage).

According to another embodiment, the recording system can intraoperatively provide a current/live 3D recording which, on the one hand, is output via the display device and, on the other hand, is stored in the annotation database in the case of an annotation instruction.

In particular, a parameter of a transparency between 0 and 1 (viz. a continuous value between 0% and 100%) and, thus, between hiding and displaying may be deposited for each annotation object in the annotation database, and the control unit may be adapted to display, together with the current 3D recording, all annotation objects corresponding to their transparency at least in the correct position, in particular in the correct pose, in the superimposed display.

According to one embodiment, the control unit can be adapted to vary a threshold for the display of outlines/contours, in particular on the basis of a manual input, to mark an area for the user as required and to shift boundaries of an area corresponding to the set threshold.

In particular, the head of the patient can be fixed with respect to the robot base of the robot and/or the robot system. This is common especially for image-based navigation systems. As an alternative, the patient can be tracked by the same positioning system, in particular tracking system.

According to another embodiment, the navigation system, and in particular the recording system, may be adapted to perform a camera calibration in order to provide the control unit with information, in particular of a three-dimensional pyramid, as to how the collected annotation objects move with respect to a change of the zoom/(the zoom step) or the focus/focal width so that the control unit is adapted to vary the camera settings during a method. In other words, the navigation system requires a positioning system (in particular a robot arm, an optical tracking system, an electromagnetic tracking) with high repeatability of the positioning in space and a (primary) camera system with high repeatability of zoom and focus/focal width, why it is of advantage when the camera system is also calibrated.

Preferably, an incremental number/a marker ID, in particular of a tracked pointing device (navigation pointer/pointer) and/or intensity information of a fluorescence recording and/or a convex envelope of trajectories and/or a spherical distance field can be stored as associated data of the annotation object in the annotation database. In other words, the following data can be deposited in particular as associated data of annotation objects: incremental number/marker ID, such as of a pointing device/computer vision tracked instrument; intensity information such as of a fluorescence recording; convex envelopes, e.g. of trajectories; a spherical distance field, e.g. from a neuro-monitoring. Annotations can specifically be as follows:
physiological information, in particular a nerve signal and/or a histology;
geometrical objects, in particular a point, a circle, a line, a trajectory and/or a contour;
and/or secondary image information, in particular a fluorescence. The different display modalities can be shown by the navigation system of the present disclosure in the (current) 3D recording where they are actually located, viz. in the correct position, in particular in the correct pose.

Preferably, the following technique can be used as a manual labeling technique: focus of the recording system, in particular of the microscope (1D object with X-Y-Z coordinates in the global coordinate system, said point in space can be appropriately labeled); a cursor/a mouse pointer in the live image of the display device (with X-Y coordinates and an offset relative to the focus in the image plane as Z coordinate); a navigated point or instrument such as a navigation pointer/pointer; computer vision techniques to recognize edges or tissue patterns; an origin of blood and a flow direction of the blood over time (ML recognized) to detect the source of bleeding.

In particular, also an automatic labeling technique can be used as annotation technique, in particular: a neuromonitoring and/or a fluorescence imaging (e.g. DUV400, DFS560, DIR800) and/or other optical measurement processes such as OCT or the like.

In particular, the navigation system can deposit, as an annotation object, a 3D video recording (limited in time), in particular having a length of substantially five seconds so that a moving image/a video is stored as annotation object. In other words, in particular a 4D annotation object (viz. 3D with a time-variable component) can be stored and accordingly reproduced again at a further point in time. In particular, this video recording can originate from an ICG.

Preferably, the surgical navigation system can further include an overview camera which is aligned and adapted to detect and provide in a computer-readable form an environment of the surgical area. In other words, additional camera systems can support an automatic generation of annotation objects.

Preferably, the navigation system may be adapted to perform an intra-image/intra-waypoint annotation. With the so-called intra-image/intra-waypoint annotation, the user moves the recording system to the desired position and adjusts the camera to the desired parameters with respect to the zoom and the focus (zoom step and focus/focal width). Now it is possible to identify annotation objects within the 2D recording manually or automatically. This category requires a robot system with high reproducibility and a primary recording system/camera system with high repeatability of zoom and focus. In particular, the focus requires the robot head, in particular a robot end effector, to move also in an appropriate pose relative to the original pose (a waypoint with stored camera parameters).

In particular, a specific camera calibration can supply information (three-dimensional pyramid) about how the collected annotation objects move with respect to a change of the zoom step or the focus/focal width. This allows the user to vary the camera settings during the process. Conventional annotation systems do not admit such flexibility during the process.

Preferably, the navigation system may be adapted to perform a spatial annotation. With the spatial annotation, the user moves the recording system (the stereoscopic recording system/camera system) to the desired position and adjusts the camera to the desired parameters with respect to the zoom step and the focus/focal width. Now it is possible to identify annotation objects within the 2D recording/the 2D image manually or automatically. Preferably via a specific camera calibration (stereoscopically), the spatial 3D positions of the labeling objects can be derived in the coordinate system of the positioning system. This category of the spatial annotation requires a robot system with high reproducibility and a recording system with high repeatability of zoom and focus/focal width. The focus requires the robot head, and, in particular the robot end effector, to move even in a posture or pose that is close to the original posture (i.e., a similar position and orientation within a tolerance; waypoint with stored camera parameters). Moreover, there is required in particular a specific camera calibration for the 3D reconstruction as well as a hand-eye calibration to transmit the data of the 3D reconstruction into the coordinate system of the positioning system. This enables the user to move the camera and to freely vary the camera settings. The visualization of the annotation objects can be visualized taking the adapted postures and camera settings into consideration. This method, too, can preferably be supported by computer vision algorithms by tracking corresponding regions when the zoom and the focus/focal width are varied and when the robot is moving.

In particular, the following user interactions can be realized by the navigation system, and/or the navigation system can be adapted to carry out the following functions: activating and deactivating the annotation objects (by corresponding entries in the annotation database, for example), supplemented by the live view; shifting a threshold for the display of outlines/contours (such as tumor boundaries) of point clouds of regular samples with appropriate intensity (e.g. a fluorescence image); user-guided segmenting of structures by simple plotting (based on so-called max-cut-min-flow).

In particular, a free movement of the recording system (camera head) can take place by an extension of the spatial annotation objects, which requires in particular a calibration. The annotation object is extended with respect to the fixed surgery site.

Preferably, in the superimposed display also at least one direction can be displayed or shown concerning stored annotations, in particular during an (automatic or manual) movement of the robot arm.

Preferably, a measurement (in mm) of annotation objects can be carried out for documentation and can be stored as data associated with the annotation object in the annotation database.

Further preferred, a focus and a zoom can be stored and/or appropriately set for spatial annotation objects, in particular on a contour of a fluorescence image.

In particular, a (1D or 2D) heat map can be added as annotation object to display target or risk structures more clearly.

An important part of the navigation system is constituted by the visualization of the detected and processed annotations. The visualization of the annotation objects can preferably be adapted so that the superposition of the objects does not interfere with inserted instruments (such as a masking-similarly to a depth of field blur of a background). As a result, the visualization is improved for the surgeon and the impression is strengthened that the annotation object is connected to the surgery field of the patient and does not only represent a simple overlay. For example, in one embodiment the control unit can be adapted to display the annotation object in such a blurred manner that the surgical instrument seems to lie there above (masked). The annotations preferably can also show through different focus images (1-2 recording planes to the top or the bottom) with a certain degree of transparency.

In particular, the navigation system may be adapted to mark detected spatial annotations as invalid when the underlying anatomy has been changed by a surgical procedure. This can be achieved by comparing the reference image of the annotation to the current live view of the camera.

Also, the navigation system, in particular the control unit, preferably may be adapted to hide the visualization of the spatial annotation object, if the visualizing error increases after a movement of the microscope head.

Preferably, the navigation system may also be adapted to deposit a biopsy or biopsy information as annotations. In particular, is it easily possible to automatically generate surgical documentations when annotation points also supply diagnostic information, in particular from biopsies.

In particular, spatial annotation objects can be used to register preoperative CT/MRI data in order to obtain additional support during the surgery.

As regards the surgical navigation method, the objects of the present disclosure are achieved by the fact that a navigation method is provided having an annotation function for inputting position-related visual medical annotations and recordings and for outputting the medical annotations during a surgical procedure on a patient, in particular for a surgical navigation system according to the present disclosure, comprising the steps of: recording a (current) 3D recording by a stereoscopic recording system arranged on or in a robot head, in particular a stereo camera, particularly preferred a stereo microscope, comprising at least two recording units spaced apart from each other, and providing a control unit with the 3D recording together with recording parameters; detecting and tracking a pose of the recording system by a tracking system and providing the control unit with the pose; when an annotation instruction is detected, determining a pose of the 3D recording on the basis of a pose of the recording system and recording parameters; storing an annotation object with at least one section of the 3D recording and the recording parameters in an annotation database of an annotation system with a global coordinate system; and outputting a superimposed display with integration of the at least one annotation object in the correct position, in particular in the correct pose, via a display device.

As regards the computer-readable storage medium and a computer program, each of the objects is achieved by the latter comprising instructions which, when executed by a computer, cause the computer to carry out the method steps of the navigation method of the present disclosure.

Any disclosure in connection with the surgical navigation system of the present disclosure is also applicable to the navigation method of the present disclosure, and vice versa.

### BRIEF DESCRIPTION OF THE DRAWINGS

In the following, the present disclosure will be illustrated in detail on the basis of preferred embodiments with the help of Figures, wherein:

FIG. 1 shows a schematic view of a navigation system according to prior art in which different sources of error are indicated;

FIG. 2 shows a perspective view of a surgical navigation system according to a preferred embodiment of the present disclosure which can display an annotation in a coherent view;

FIG. 3 shows a further perspective view of a surgical navigation system according to another preferred embodiment which detects an annotation object with depth information;

FIG. 4 shows a perspective view of the navigation system of FIG. 3 which, in a second position, detects or outputs the adapted display of the annotation;

FIG. 5 shows a further perspective view of the navigation system of FIGS. 3 and 4 in which an annotation is moved out of a detection area;

FIG. 6 shows a schematic view of an exemplary 3D recording with circular and punctiform annotations;

FIG. 7 shows a schematic view of an exemplary 3D recording with a contour annotation;

FIG. 8 shows a schematic view of an exemplary 3D recording with a trajectory annotation;

FIG. 9 shows a schematic view of an exemplary 3D recording with a fluorescence annotation;

FIG. 10 shows a further schematic view of an exemplary 3D recording with a fluorescence annotation;

FIG. 11 shows a schematic view of an exemplary 3D recording with a tracked instrument and associated distance fields as annotations;

FIGS. 12 and 13 show a schematic view of an exemplary 3D recording with movement vectors of a vascular fluorescence (ICG) as stored annotations;

FIGS. 14 and 15 show a schematic view of an exemplary 3D recording with a biopsy as annotation;

FIG. 16 shows a schematic view of an exemplary 3D recording with differently set thresholds for a detection of a boundary as annotation;

FIG. 17 shows a schematic view of an exemplary 3D recording with a deposited dimension in mm as annotation;

FIG. 18 shows a schematic view of an exemplary 3D recording with an augmented heat map as annotation;

FIG. 19 shows a schematic view of an exemplary 3D recording with a reproducible 4D annotation (video); and

FIG. 20 shows a schematic view of an exemplary 3D recording with a tracked instrument and an annotation moved into the background so that the instrument appears in the foreground.

The Figures are schematic and are intended to serve for the comprehension of the invention only. Like elements are provided with the same reference signs. The features of the different embodiments can be exchanged.

### DETAILED DESCRIPTION

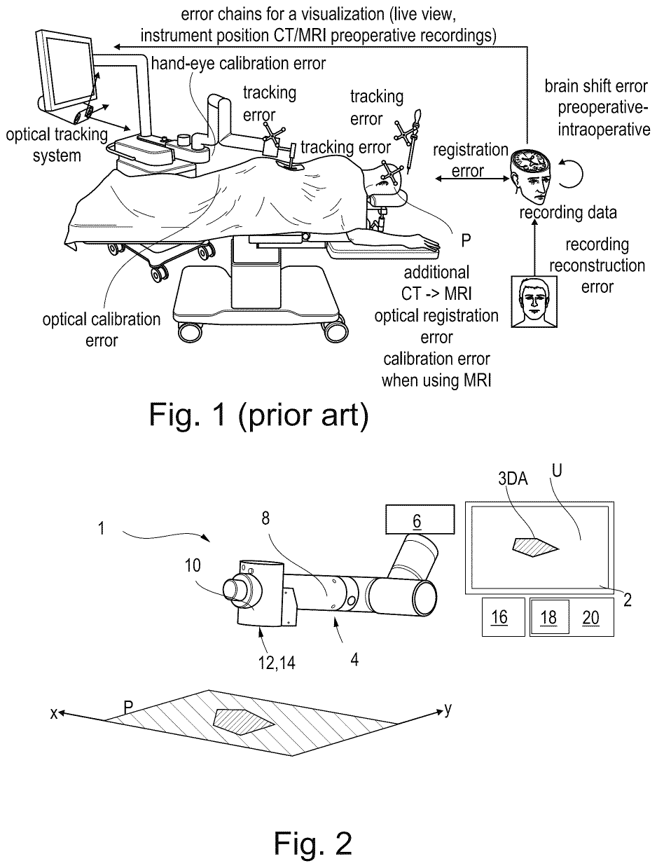

FIG. 1 shows in a perspective schematic view a surgical navigation system according to prior art. In this case, a plurality of sources of error occur which have a negative influence on precision. Instruments are tracked via attached rigid bodies (infrared markers) by the tracking camera, wherein tracking errors having a certain error tolerance occur for each individual rigid body. Also, a patient is tracked via a rigid body with an accompanying tracking error. Further, an error may occur in the recording reconstruction and in a change of the anatomy with respect to the preoperative recording data, for example by a tissue shift like a so-called brain shift. Another source of error may be a hand-eye camera calibration or an optical calibration. These errors as well as error chains must be avoided or at least minimized to enable even better and more precise navigation.

FIG. 2 illustrates a perspective view of a surgical navigation system 1 (hereinafter only referred to as navigation system) having an annotation function. Said annotation function serves to intraoperatively integrate position-related visual medical annotations and recordings and to use them for guide and a surgical procedure on a patient P (indicated here).

In order to be able to visually display the input annotations, the navigation system 1 includes a display device 2 in the form of a surgery monitor in order to visually output the medical annotations for qualified medical personnel, in particular a surgeon.

The navigation system 1 further comprises a robot 4 as a positioning system. Concretely, the robot has a stationary robot base 6 and a multi-link robot arm 8 movably connected to the robot base 6 which can be actively controlled and moved/displaced. A terminal robot head 10 which can be rotated at least relative to a longitudinal axis of the last segment/link of the robot arm 8 is movably connected to an end portion of the robot arm 8.

In the robot head 10, a stereoscopic recording system 12, in particular a stereo camera, in the present case a stereo microscope, comprising at least two recording units 14 spaced apart from each other, in this case recording cameras, is received which records a 3D recording 3DA of a structure or surface via two optical systems each having a downstream sensor. Said 3D recording 3DA is provided together with recording parameters, in this case, in particular camera parameters, in a computer-readable form.

For tracking the robot head 10, the navigation system 1 comprises a tracking system 16 which, in the present embodiment, is configured as a mechanically sensory tracking system 16 and, via the robot arm configuration, detects a pose of the robot head 10 relative to the robot base 6 and, resp., relative to a coordinate system of the robot 4 and indirectly, via the pose of the robot head 10, detects and spatially tracks the pose of the recording system 12. Said detected pose of the recording system 12 is provided in a computer-readable form.

For data management of annotations, the navigation system 1 comprises a central internal annotation system 18 having a storage unit (not shown here) in which a digital annotation database with one single global coordinate system is stored, in the present case the global coordinate system being the coordinate system of the robot 4. In said annotation database, a new annotation object can be entered as a list entry (similarly to a SQL database), and data associated with the annotation object can be stored.

A particularly adapted control unit 20 (annotation system 18 is a part/sub-unit of the control unit 20) is configured to process the provided 3D recording 3DA together with the recording parameters and the provided pose of the recording system. When an annotation object is deposited in the annotation database, based on the provided recording parameters and the pose of the recording system, the control unit 20 can initially determine a pose of the 3D recording 3DA in space, that is, a pose relative to the central global coordinate system. Said deposited annotation object and, resp., the data of the annotation object then is output, when it comes within the field of view of the 3D recording, in a superimposed display in the correct position, in particular in the correct pose. For example, an image, a text or other medical data for a surgical procedure can be deposited as an annotation object, and the image can be displayed in the 3D recording in the correct pose or the text can be displayed in the 3D recording in the correct position.

Further, the control unit 20 is particularly adapted to process the provided 3D recording 3DA together with the recording parameters and the provided pose of the recording system and in the case of an annotation instruction, for example via the touch display of the surgery monitor as display device or automatically based on an internal annotation program, to initially determine a pose of the 3D recording in space, based on the provided recording parameters and the pose of the recording system, i.e., a pose relative to the central global coordinate system. In other words, the pose of the 3D recording is finally determined starting from the robot base 6 via the robot head 10, via the recording system 12 and the optical parameters. Said pose is transferred into the central global coordinate system.

In the annotation database of the annotation system, at least a section of the 3D recording, such as of an area of interest, for example a tumor, is stored together with the recording parameters in the global coordinate system in the specific position, and here even the specific pose, as an annotation object. In particular when the navigation system 1 does not only have one robot 4 but plural robots 4 including robot arms, important data for the surgical procedure can be stored centrally, wherein one (single) global coordinate system centrally records all annotations. Said global coordinate system can be imagined to be a three-dimensional space with a Cartesian coordinate system that has stored different types of annotations which were recorded intraoperatively at different X-Y-Z positions.

At a later point in time, the surgeon then can output the previously recorded annotation object via the display device 2 in a superimposed display U in a live display of the current recording of the recording system 12 at least in the correct position, such as a point in space fixed by means of a navigation pointer, or in the correct pose, such as the 3D recording of a surface. A surgical navigation is clearly improved in this way.

FIG. 2 illustrates a coherent view which means that, at a first point in time in a first pose of the recording system, an annotation instruction was given, the annotation was stored correspondingly with the associated recording data, i.e., substantial parameters of the camera setting such as the focus and the zoom as well as the robot configuration (configuration of the links of the robot arm 8 relative to one another and to the robot base 6) in the central annotation database in the global coordinate system at the correct position with additional information about the orientation, and, after the first point in time, the robot was moved and a surgical procedure was continued as appropriate.

If the surgeon intends to return to the first annotation, he/she selects the corresponding annotation by means of an output instruction, by manual input or activation of the touch display of the surgery monitor, and the control unit controls, on the basis of the data deposited concerning the annotation object, the robot arm 8 into the configuration intended for this purpose and, thus, into the same pose of the recording system 12 as at the first point in time, focuses the camera corresponding to the stored camera settings and outputs a superimposed display including, on the one hand, the current 3D recording 3DA and, on the other hand, the section of the stored 3D recording 3DA, such as a juxtaposed display or a superposition by means of transparency, to visually present a change over time to the surgeon.

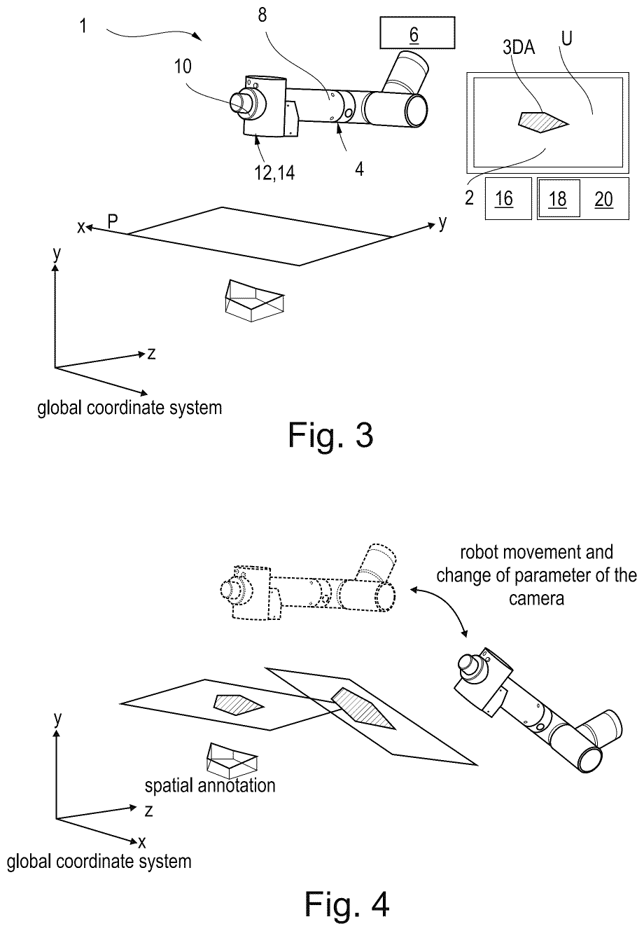

FIGS. 3 and 4 illustrate a perspective view of a surgical navigation system 1 of a further preferred embodiment. Said embodiment only differs by the fact that the control unit is adapted to perform a spatial annotation, wherein the control unit 20 is adapted to display, even from a second pose that is different from a first pose (see FIG. 4 first pose shown in broken lines, second pose shown in solid lines), a spatial display, such as by corresponding matrix transformations, of the previously recorded annotation object. In doing so, the central global coordinate system with deposited annotation objects in connection with image analysis and recording processing procedures helps to display the annotation in the correct pose in the second pose of the recording system 12.

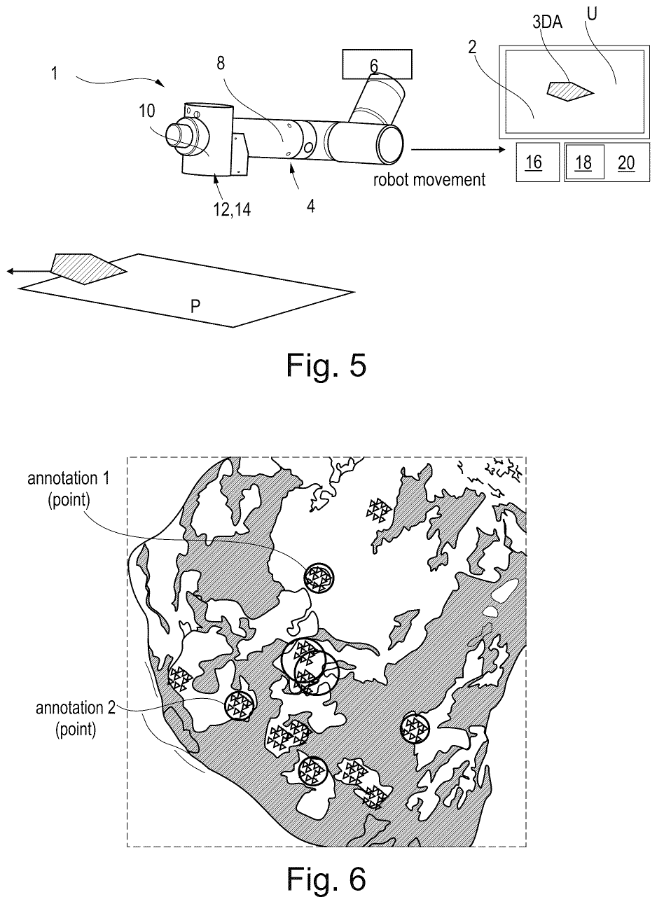

FIG. 5 schematically illustrates the situation when the robot 4 is moved and the object to be annotated moves out of the field of view (e.g., in the coherent view).

FIG. 6 schematically shows an example of an annotation stored in the annotation database. A point in space of the global coordinate system with X, Y and Z coordinates is deposited as an annotation object having a corresponding labeling that can also be shown in a superimposed display U as required.

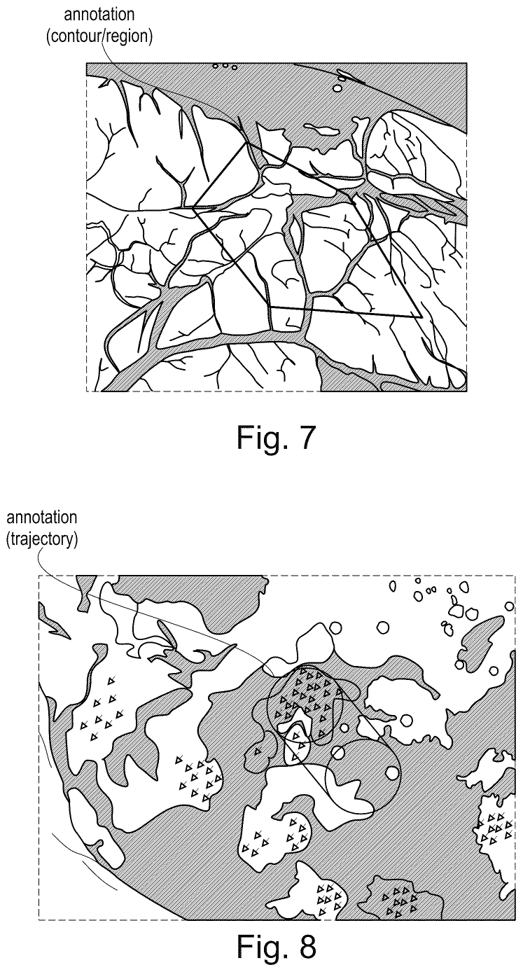

FIG. 7 illustrates a further alternative of a stored annotation in which an area of a tissue surface was marked (in color) as a pentagon. In a surgical procedure, said area can also be displayed to the surgeon.

FIG. 8 illustrates a further annotation in the form of a trajectory which is also displayed in the tissue in order to provide the surgeon with a guide.

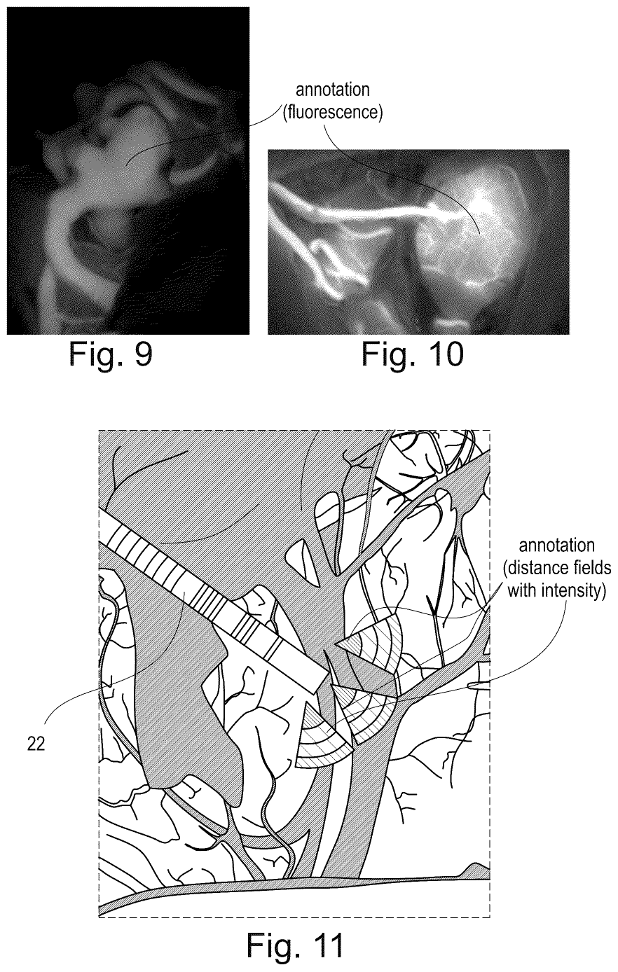

FIGS. 9 and 10 illustrate 3D records of a tissue structure having a deposited fluorescence as an annotation.

In FIG. 11, an instrument 22, in this case a pointer, having ring marks on the instrument 22 itself as well as distance fields at the distal tip of the instrument 22 is shown as a further configuration of annotations.

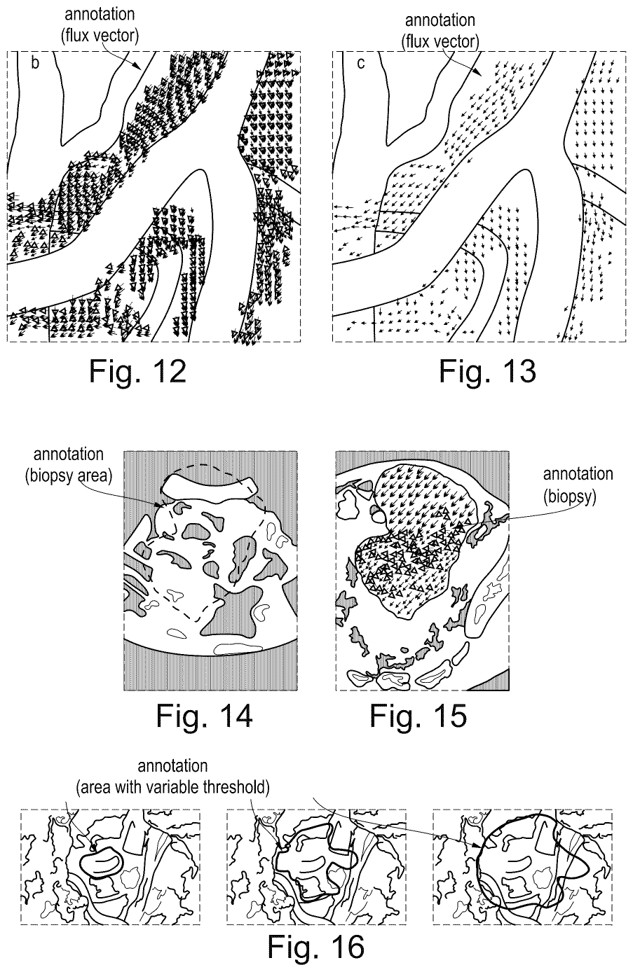

FIGS. 12 and 13 illustrate, in a deposited 3D recording 3DA, flux vectors as annotations or annotation objects.

FIG. 14 illustrates an area in which a biopsy was taken, and FIG. 15, in turn, shows biopsy results having different areas in a schematic view.

FIG. 16 illustrates (from the left to the right) a change of a boundary/limit of an area in which a threshold is continuously reduced by the user. In other words, the user can predetermine a threshold, and a boundary is visualized around a tissue area based on said threshold. This is another alternative of an annotation.

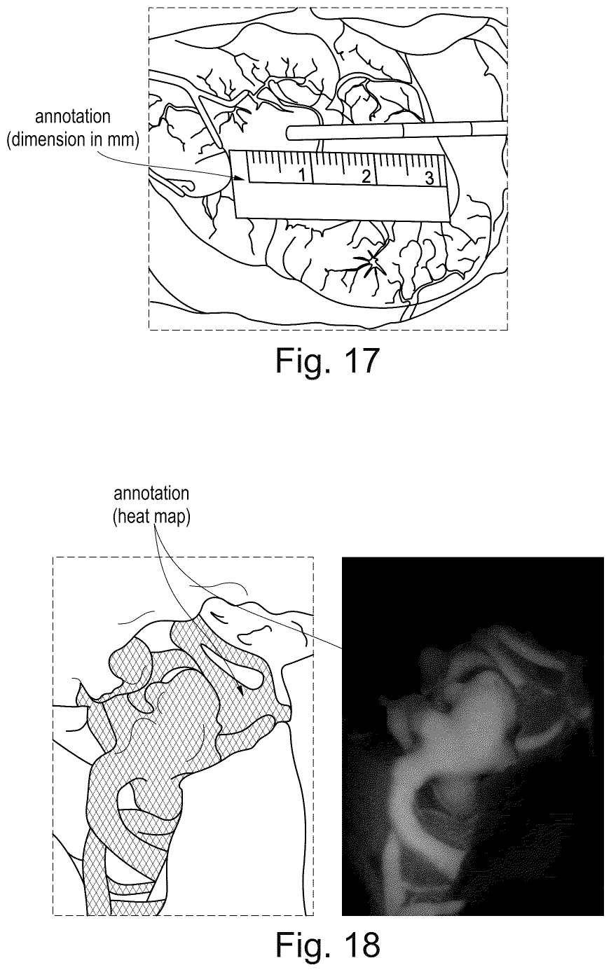

FIG. 17 shows a dimension in mm as an annotation. Accordingly, a sterile measurement instrument in the form of a ruler was positioned inside the tissue and a 3D recording was performed. In this way, at a later point in time, the dimension can be displayed where needed, if the recording system is in a similar situation again, without the measurement instrument having to be physically integrated again.

FIG. 18 illustrates another alternative of an annotation in the form of a heat map.

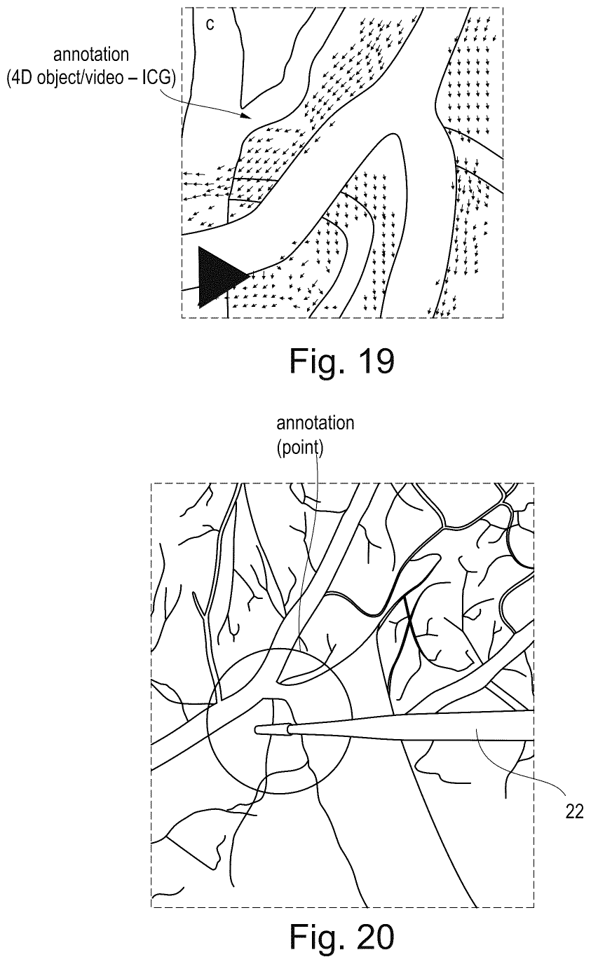

FIG. 19 illustrates a video of a flow as an annotation so that also moving images can be reproduced.

FIG. 20 illustrates an instrument 22 in the current recording, while a marker point, in this case shown by a circle, is displayed as an annotation in the background of the instrument. The control unit 20 is adapted to recognize instruments 22 and to put them to the front in the plane so that the annotation does not cover the instrument 22.

### LIST OF REFERENCE SIGNS

1 surgical navigation system
2 display device
4 robot
6 robot base
8 robot arm
10 robot head
12 recording system
14 recording unit
16 tracking system
18 annotation system
20 control unit
22 instrument
P patient
3DA 3D recording
U superimposed display

1.-15. (canceled)

16. A surgical navigation system having an annotation function for inputting medical annotations or recordings that are position-related, and/or for outputting medical annotations during a surgical procedure on a patient, the surgical navigation system comprising:
a display device for a visual output of the medical annotations;
at least one robot as a positioning system, the at least one robot comprising a robot arm movably connected to a robot base and a robot head arranged at an end of the robot arm,
a stereoscopic recording system arranged on or in the robot head, the stereoscopic recording system comprising at least two recording units spaced apart from each other in order to perform a 3D recording of a structure or surface relative to the stereoscopic recording system and to provide the 3D recording together with recording parameters in a computer-readable form;
a tracking system adapted to detect and track a pose of the stereoscopic recording system and to provide the pose of the stereoscopic recording system in a computer-readable form;
an annotation system comprising a storage unit in which an annotation database with a global coordinate system for annotation objects is stored; and
a control unit adapted to process the 3D recording together with the recording parameters and the pose of the stereoscopic recording system and, based on the recording parameters and the pose of the stereoscopic recording system, to determine a pose of the 3D recording in the global coordinate system and to output at least one annotation object of the annotation database in the 3D recording at least in a correct position via the display device in a superimposed display and to improve navigation.

17. The surgical navigation system according to claim 16, wherein the control unit is adapted, in response to an annotation instruction, to determine a pose of the 3D recording based on the recording parameters and the pose of the stereoscopic recording system, and to store, as the at least one annotation object, at least a section of the 3D recording together with the recording parameters in the annotation database of the annotation system in the global coordinate system in a specific position in order to output the at least one annotation object at a later point in time at least in the correct position via the display device in the superimposed display and to improve navigation.

18. The surgical navigation system according to claim 17, wherein the control unit is adapted to display a warning message in the superimposed display when the section of the 3D recording deviates beyond a preset tolerance relative to a current 3D recording.

19. The surgical navigation system according to claim 16, wherein a robot arm configuration of the at least one robot and a camera setting of the stereoscopic recording system are stored in the annotation database as recording parameters in order to move the at least one robot at a later point in time of the surgical procedure into a specific position and orientation with a corresponding camera setting and to output the 3D recording and a current 3D recording as a coherent reference in the superimposed display to visualize a change over time.

20. The surgical navigation system according to claim 16, wherein a zoom and a focus detected as camera settings of the stereoscopic recording system are stored as recording parameters so that at least a position of a segment of the 3D recording is precisely determined via the pose of the stereoscopic recording system tracked by the tracking system and via the zoom and the focus.

21. The surgical navigation system according to claim 16, wherein the control unit is adapted to move, in response to an output instruction concerning the at least one annotation object of the annotation database, the stereoscopic recording system into the pose stored relative to the at least one annotation object and to output a current 3D recording and the at least one annotation object in the superimposed display via the display device.

22. The surgical navigation system according to claim 16, wherein
preoperative 3D recording data are further stored in the storage unit;
the surgical navigation system is adapted to register the patient relative to the preoperative 3D recording data; and
the control unit is adapted to display also the preoperative 3D recording data for further support in the superimposed display relative to the 3D recording.

23. The surgical navigation system according to claim 16, wherein the control unit is adapted to detect an instrument in a current 3D recording and to put the instrument in a front plane in the superimposed display, while the at least one annotation object is shown in a rear plane, so that in the superimposed display the at least one annotation object does not interfere with the instrument.

24. The surgical navigation system according to claim 16, wherein the stereoscopic recording system intraoperatively provides a current 3D recording that is output via the display device and, in case of an annotation instruction, is stored in the annotation database.

25. The surgical navigation system according to claim 16, wherein a parameter of a transparency between 0 and 1 and, thus, between hiding and displaying is deposited for each annotation object in the annotation database, and the control unit is adapted to display all annotation objects corresponding to their transparency together with a current 3D recording at least in a correct position in the superimposed display.

26. The surgical navigation system according to claim 16, wherein the control unit is adapted to vary a threshold for indicating outlines/contours as an annotation in order to mark an area for a user as required.

27. The surgical navigation system according to claim 16, wherein the stereoscopic recording system is adapted to perform a camera calibration in order to provide the control unit with information about how collected annotation objects move with respect to a change of a zoom or a focus in order to enable the control unit to vary camera settings during a movement.

28. The surgical navigation system according to claim 16, wherein an incremental ID and/or an intensity information of a fluorescence recording and/or a convex envelope of trajectories and/or a spherical distance field are stored as associated data of annotation objects in the annotation database.

29. A surgical navigation method having an annotation function for inputting medical annotations and recordings that are position-related and for outputting the medical annotations during a surgical procedure on a patient, the surgical navigation method comprising the steps of:
recording a 3D recording by a stereoscopic recording system arranged on or in a robot head, comprising at least two recording units spaced apart from each other, and providing a control unit with the 3D recording together with recording parameters;
detecting and tracking a pose of the stereoscopic recording system by a tracking system and providing the control unit with the pose;
when detecting an annotation instruction, determining a pose of the 3D recording from the pose of the stereoscopic recording system and the recording parameters;
storing an annotation object with at least one section of the 3D recording and the recording parameters in an annotation database of an annotation system with a global coordinate system; and
outputting a superimposed display while integrating the annotation object in a correct position via a display device.

30. A computer-readable storage medium comprising instructions which, when being executed by a computer, cause the computer to carry out the surgical navigation method according to claim 29.
